(19) United States
(12) Patent Application Publication
(10) Pub. No.: US 2023/0364313 A1
(43) Pub. Date: Nov. 16, 2023

(54) ACCESS SYSTEM FOR A MEDICAL DEVICE FOR DRAWING A MEDICAL LIQUID, MONITORING SYSTEM COMPRISING SUCH AN ACCESS SYSTEM, AND MEDICAL TREATMENT DEVICE COMPRISING SUCH A MONITORING SYSTEM

(71) Applicant: FRESENIUS MEDICAL CARE DEUTSCHLAND GMBH, Bad Homburg (DE)

(72) Inventors: Joachim NOACK, Bad Neustadt (DE); Klaus SAUER, Reuchelheim (DE); Peter KLOEFFEL, Nuedlingen (DE); Rene BAUER, Hambach (DE); Matthias FEHLER, Leinach (DE); Markus THEN, Schwarzach (DE); Burkard KELLER, Wuerzburg (DE); Achim EBERLEIN, Schweinfurt (DE); Oliver BOND, Heidenfeld (DE)

(73) Assignee: FRESENIUS MEDICAL CARE DEUTSCHLAND GMBH, Bad Homburg (DE)

(21) Appl. No.: 18/030,079

(22) PCT Filed: Oct. 5, 2021
(86) PCT No.: PCT/EP2021/077422

§ 371 Date: Apr. 4, 2023

(30) Foreign Application Priority Data
Oct. 7, 2020 (DE) 10 2020 126 224.8

(51) Int. Cl. A61M 1/16; A61M 39/10

(52) U.S. Cl. CPC A61M 1/168; A61M 39/10

(57) ABSTRACT

The invention relates to an access system 1 for a medical device, which system has a housing body 21 in which an inner pipe portion 22 for transporting a medical fluid is formed, which portion is enclosed by an outer pipe portion 24 so as to form an empty space 23 for receiving a disinfectant fluid, the housing body 21 having an opening 25 that can be closed by a closure element. The access system 1 according to the invention is characterised in that a measuring electrode 30 and at least one counter electrode 31, 32 are arranged in the housing body 21 such that the measuring electrode 30 interacts with the counter electrode via the empty space 23. The measuring electrode 30 allows an electrical signal to be input such that a current flowing between the measuring electrode and the counter electrode or a voltage applied between the measuring electrode and the counter electrode can be evaluated. On the basis of the evaluation of the current or voltage, the presence or absence of a fluid or moisture in the empty space 23 can be inferred and/or a conclusion can be made as to whether a particular fluid is present in the empty space 23. The invention also relates to a monitoring system 2 having such an access system 1, to a medical treatment device 1 having such a monitoring system 2, and to a method for monitoring an access system for a medical device.

The invention relates to an access system for a medical device, which system has a housing body in which an inner pipe portion for transporting a medical fluid is formed, which portion is enclosed by an outer pipe portion so as to form an empty space for receiving a disinfectant fluid, the housing body having an opening that can be closed by a closure element. The invention also relates to a monitoring system comprising such an access system and to a medical treatment device comprising such a monitoring system. The invention also relates to a method for monitoring an access system for a medical device.

For supplying or withdrawing a fluid, access systems are used in medical technology that allow sterile connection of a hose line in order to be able to supply or withdraw a fluid. Access systems of this kind are also referred to as ports.

In haemodialysis machines that are set up for haemodiafiltration, the patient's blood is thinned by adding substituate. The substituate can be provided in containers or obtained in the dialysis machine from dialysate via a sterile filter. Haemodialysis machines are known which have an access system to which a hose line is connected in order to be able to feed the substituate provided by the dialysis machine to the extracorporeal circuit. When not in use, the access system is tightly closed by a closure cap in order to avoid contamination. Before connecting the hose line, the closure cap is removed. For the access system, care must be taken that germs or pathogens that can adhere to the access system in daily practice do not get into the patient's blood. Therefore, the access system is generally flushed with a disinfectant solution. The disinfectant solution can, for example, be a heated and therefore germicidal fluid (dialysate, substituate, RO water), which can be provided by the dialysis machine. Alternatively, a chemical disinfectant solution can also be used. It is essential that all parts of the access system that can come into contact with the patient are flushed with the disinfectant solution in order to exclude contamination. After disinfection, no residues of the disinfectant solution should remain in order to reliably prevent the blood from coming into contact with the disinfectant solution. In general, with the aid of the invention, residues of any conductive fluids can be identified, for example also of substitution solution, which can also be used, inter alia, for flushing and filling the extracorporeal blood circuit.

The access system can be disinfected during a shift before each dialysis treatment. For reasons of cost and time, however, it is also possible that disinfection in the dialysis centre only takes place before or after each shift (e.g. at night). It is therefore of particular interest to avoid contamination that can result from handling the device during a shift and, if necessary, to carry out further disinfection or to prevent further treatment. It is also of interest to determine during disinfection whether the critical parts of the access system come into contact with disinfectant solution. It is also of interest to check the tightness of the access system. In particular, it is of interest to check the tightness of the access system during its intended use, i.e. when a substitution solution is provided.

The aim of the invention is that of providing an access system for a medical device, in particular for a dialysis machine, in particular for withdrawing a medical fluid, for example substituate, which allows reliable monitoring of its proper condition during and after disinfection. In addition, an aim of the invention is that of providing a monitoring system comprising such an access system and a medical treatment device comprising such a monitoring system, which device allows reliable monitoring of the proper condition during and after disinfection. Another aim of the invention is that of providing a method for monitoring an access system for a medical device, by means of which method reliable monitoring of the access is possible.

These aims are achieved according to the invention by the features of the independent claims. The dependent claims relate to preferred embodiments of the invention.

The access system according to the invention for a medical device has a housing body in which an inner pipe portion for transporting a medical fluid is formed, which portion is enclosed by an outer pipe portion so as to form an empty space for receiving a disinfectant fluid, the housing body having an opening that can be closed by a closure element.

The access system according to the invention is intended in particular for the withdrawal of a medical fluid. However, the access system can also be used to supply a medical fluid. As a result, the opening of the housing body can be used for withdrawing or supplying a medical fluid. The medicinal fluid can be, for example, substituate. The housing body allows the access system to be attached to the medical device, for example a medical treatment device, in particular a haemodialysis machine. If a hose line is connected to the access system, the medical fluid, for example substituate, flows through the inner pipe portion. During disinfection, disinfectant fluid flows through the empty space, which is closed in a fluid-tight manner by the closure element, and so disinfectant fluid washes around the relevant parts of the access, in particular the region around the inner pipe portion.

The access system according to the invention is characterised in that a measuring electrode and at least one counter electrode are arranged in the housing body such that the measuring electrode interacts with the counter electrode via the empty space. The measuring electrode allows an electrical signal to be coupled in or input such that a current flowing between the measuring electrode and the counter electrode or a voltage applied between the measuring electrode and the counter electrode can be evaluated. In this context, the evaluation of a current and a voltage also means the measurement of a (complex) resistance (impedance or reactance measurement). On the basis of the evaluation of the current or voltage (complex resistance), the presence or absence of a fluid or moisture in the empty space can be inferred and/or a conclusion can be made as to whether a particular fluid is present in the empty space, i.e. one fluid can be distinguished from another fluid.

If, during the disinfection of the access system, the presence of a fluid in the empty space is inferred, it can be assumed that the empty space is at least partially filled with disinfectant. A plurality of counter electrodes can be provided for monitoring the complete filling or for identifying only partial filling of the empty space (fill level) with disinfectant fluid, the counter electrodes each being associated with a particular region or portion of the empty space.

After the access system has been disinfected, it can be checked whether moisture is still present in the empty space, which moisture forms a conductive connection between the measuring electrode and the counter electrode, i.e. whether moisture is still present in the port. It is assumed that a dry port is generally sterile, since practice has shown that most germs are bound to moisture.

In a preferred embodiment, the access system comprises a connector which can be inserted into the opening for withdrawing or supplying the medical fluid, which connector has a pipe portion extending into the empty space, which portion can be connected in a fluid-tight manner to the inner pipe portion of the housing body, the connection point between the pipe portion of the housing body and the pipe portion of the connector being in the empty space.

The tightness of the access system can be checked during operation. If fluid is found in the empty space, which is inherently dry, it can be concluded that there is a leak at the connection point between the pipe portions of the housing body and of the connector, which connection point is located in the empty space.

In another preferred embodiment, the measuring electrode is a pin which is electrically insulated from the housing body and extends into the empty space. The pin can be provided with an electrical connection on the housing side.

In a particularly preferred embodiment, the counter electrode is formed by at least one part of the inner pipe portion. This embodiment is advantageous for the identification of fluid, in particular substituate, which can escape at the connection point between the pipe portions of the housing body and of the connector. In this embodiment, at least one part of the inner pipe portion can consist of a conductive material or at least one part of the outer wall of the inner pipe portion can be provided with a coating made of a conductive material.

In a further particularly preferred embodiment, the at least one counter electrode is formed by at least one part of the outer pipe portion. This embodiment is advantageous if the filling of the empty space with disinfectant fluid is to be identified. In this embodiment, at least one part of the outer pipe portion can consist of a conductive material or at least one part of the inner wall of the outer pipe portion can be provided with a coating made of a conductive material. Depending on the fill level of disinfectant fluid in the empty space, a different resistance is set between the measuring electrode and the counter electrode. The more the empty space is filled, the more current paths are formed, and therefore the resistance decreases. If a plurality of counter electrodes are formed on the outer pipe portion, said counter electrodes can be arranged such that, depending on the fill level, particular conductive paths to the individual counter electrodes are formed. In both cases, monitoring can be carried out by corresponding evaluation of the signals, for example by a deviation from a reference value.

The access system can also have both embodiments, and therefore one or more current paths between the measuring electrode and the inner pipe portion and the outer pipe portion can be detected.

The monitoring system according to the invention, which comprises the access system according to the invention, has a means for generating an electrical signal, which means is electrically connected to the measuring electrode and to the at least one counter electrode, and an evaluation and arithmetic means which is configured such that a current flowing between the measuring electrode and the at least one counter electrode or a voltage applied between the measuring electrode and the at least one counter electrode is evaluated.

The evaluation and arithmetic means can be configured such that a current flowing between the measuring electrode and the at least one counter electrode or a voltage applied between the measuring electrode and the at least one counter electrode is evaluated such that the presence or absence of a fluid or moisture in the empty space is inferred, or can be configured such that it is identified whether a particular fluid is present in the empty space. The known evaluation methods can be used to determine the condition of the access system.

The evaluation and arithmetic means can be configured such that a control signal or reporting signal is generated when a fluid or moisture located in the empty space is inferred and/or a control signal or reporting signal is generated when a fluid or moisture located in the empty space is not inferred. The control signal or reporting signal can be used, for example, to intervene in the machine control of the medical device, for example to prevent further treatment or to give an alarm. The operating personnel can also be prompted, on a display, to carry out disinfection.

If the earthing of the counter electrode is faulty, especially if the earthing is interrupted, increased leakage currents may occur. The means for generating an electrical signal is therefore preferably configured such that an electrical signal is generated at successive time intervals. Since the measurement signal is only applied for a short time, the average current is lower than for continuous application.

For safety reasons, a coupling capacitor can also be provided between the evaluation and arithmetic means and the measuring electrode.

In another preferred embodiment, the means for generating an electrical signal has a frequency generator for generating an alternating voltage signal or alternating current signal.

The evaluation and arithmetic means can have a means for rectifying an alternating voltage signal, the evaluation and arithmetic means being configured such that the rectified alternating voltage signal (direct current voltage) is compared with a reference value. The presence of a fluid or moisture in the empty space can then be inferred if the rectified alternating voltage signal is smaller than the reference value. The fill level can be inferred on the basis of the level of the direct current voltage, i.e. on the basis of the electrical resistance.

However, it is also possible to evaluate a non-rectified alternating voltage signal/alternating current signal, which occurs upon excitation with an alternating voltage via the conductive connection between the measuring electrode and the counter electrode, which connection is formed by fluid/moisture, in order to be able to identify a particular fluid, for example substituate. The methods for evaluating the signals can be found in the prior art. In this regard, reference is made to DE 10 2010 028 902 A1.

Embodiments of the invention are explained below in detail with reference to the drawings, in which:

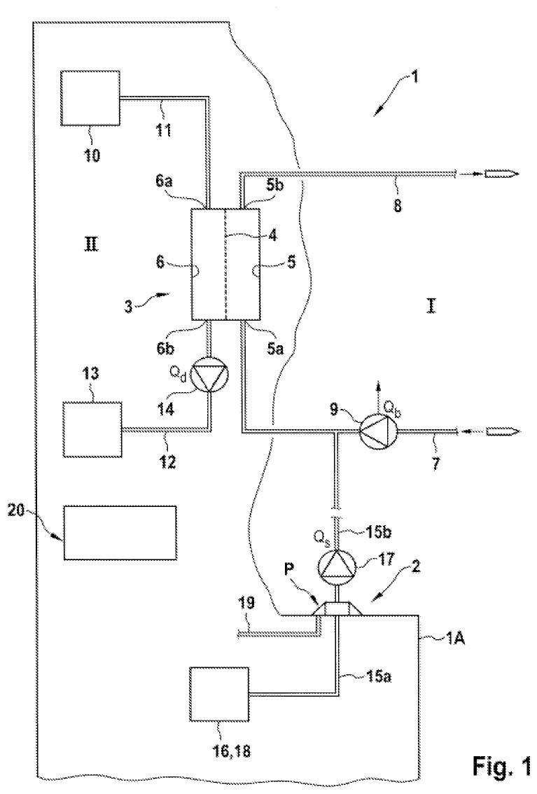

FIG. 1 is a greatly simplified schematic view of a haemodialysis apparatus according to the invention, the haemodialysis apparatus having the monitoring system according to the invention comprising the access system according to the invention;

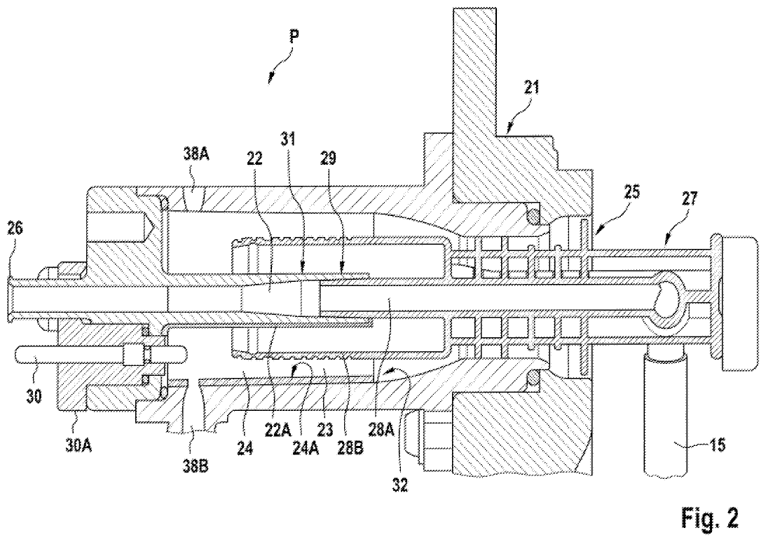

FIG. 2 is a sectional view of an embodiment of the access system according to the invention;

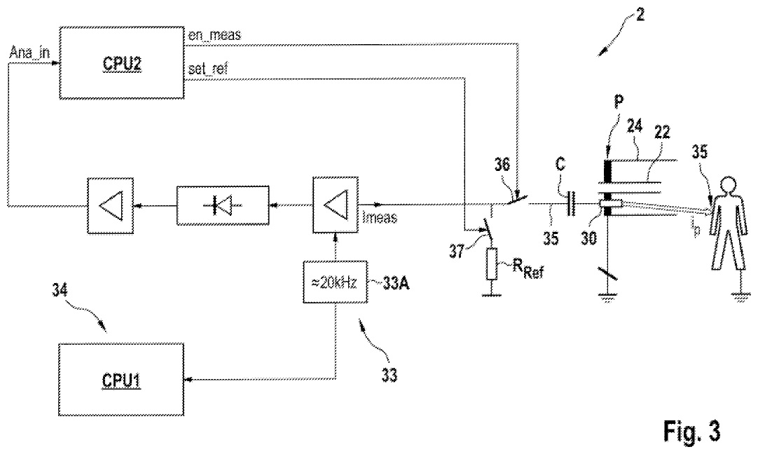

FIG. 3 shows an embodiment of the monitoring system according to the invention;

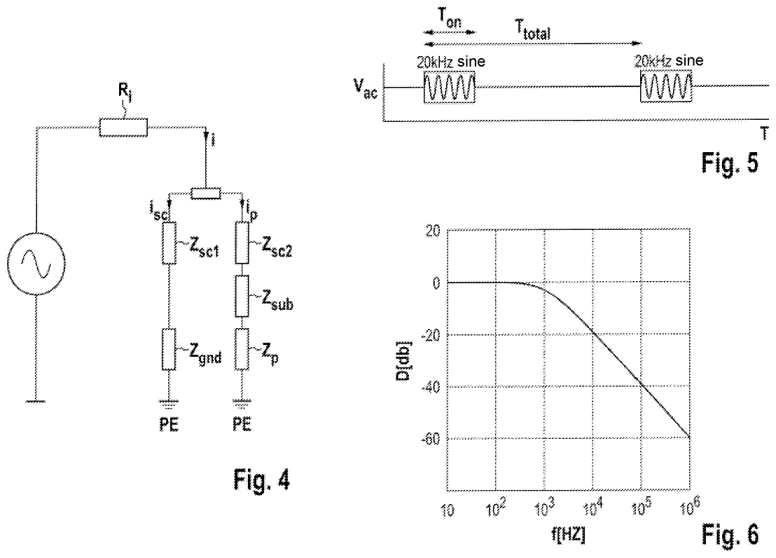

FIG. 4 shows an electrical equivalent circuit diagram to illustrate the current flow;

FIG. 5 shows the time curve of the alternating voltage signal;

FIG. 6 shows the attenuation of the alternating voltage signal as a function of the frequency; and

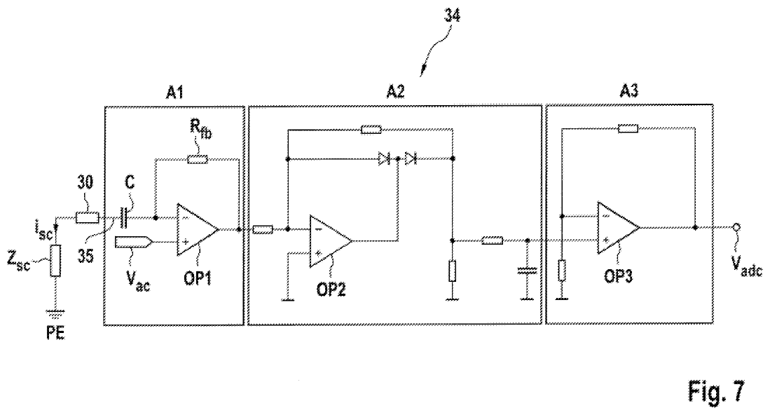

FIG. 7 shows an embodiment of the evaluation and arithmetic means of the monitoring system.

As an example of a medical treatment device 1, FIG. 1 is a greatly simplified schematic view of an extracorporeal blood treatment apparatus which has a monitoring system 2 for monitoring an access to the medical treatment device.

The present extracorporeal blood treatment apparatus is a haemo(dia)filtration apparatus which has a dialyser 3 that is separated by a semipermeable membrane 4 into a blood chamber 5 through which blood flows and a dialysis fluid chamber 6 through which dialysis fluid flows. The blood chamber 5 is part of an extracorporeal blood circuit I, while the dialysis fluid chamber 4 is part of a dialysis fluid system II of the haemo(dia)filtration apparatus.

The extracorporeal blood circuit I comprises an arterial blood line 7, which leads to the inlet 5a of the blood chamber 5, and a venous blood line 8, which branches off from outlet 5b of the blood chamber 5 of the dialyser 3. The patient's blood is conveyed through the blood chamber 5 of the dialyser 1 by means of an arterial blood pump 9, which is arranged on the arterial blood line 7. The blood lines 7, 8 and the dialyser 3 form a disposable which is intended for single use and is inserted into the dialysis apparatus for the dialysis treatment.

The fresh dialysis fluid is provided in a dialysis fluid source 10. A dialysis fluid supply line 11 leads from the dialysis fluid source 10 to the inlet 6a of the dialysis fluid chamber 6 of the dialyser 3. A dialysis fluid discharge line 12 leads from the outlet 6b of the dialysis fluid chamber 6 to a drain 13. A dialysis fluid pump 14 is connected into the dialysis fluid discharge line 12.

During the dialysis treatment, substitution fluid (substituate) can be fed to the extracorporeal blood circuit I via a substituate line 15b. In the present embodiment, the substituate line 15b is connected to a line portion of the arterial blood line 7. The substituate can be a fluid provided in a substituate source 16 and can be conveyed by means of a substituate pump 17. The substituate source 16 can be a container filled with prepared substituate. In one embodiment, the substituate can also be produced from the filtration of the dialysate, via sterile filters, from the dialysis fluid source 10 in the extracorporeal blood treatment apparatus (not shown in FIG. 1).

The substituate line 15b is part of the disposable intended for single use. To connect the substituate line 15b to the blood treatment apparatus, an access system P (port), which is only shown schematically in FIG. 1, is provided on the housing 1A of the blood treatment apparatus 1, which housing is only indicated in FIG. 1. A fluid connection 15a is provided, inter alia, for connecting the substituate source 16 to the access system P.

The access system P can be disinfected before or after a dialysis treatment or at particular time intervals, for example once a day. In the present embodiment, the disinfectant fluid for disinfecting the access system P is provided in a container 18, which can be used in place of the substituate source 16. To carry out the disinfection, the disinfectant fluid is connected to the access system P via the fluid connection 15a. During the disinfection, the access system P is flushed through with disinfectant fluid by the disinfectant fluid being conducted from the container 18 to the access system P and from there being removed again via a drain line or return line 19

The blood treatment apparatus 1 has a monitoring system 20 (only indicated in FIG. 1) for monitoring the condition of the access system.

An embodiment of the access system P (port) is described in detail below with reference to FIG. 2.

The access system P has a multi-part housing body 21 which is attached to the housing 1A of the blood treatment apparatus 1 so as to be freely accessible to the operating personnel. An inner pipe portion 22 for transporting the substitution fluid or disinfectant fluid is formed in the housing body 21. The inner pipe portion 22 is enclosed by an outer pipe portion 24 (which tapers to the right in FIG. 2) so as to form an empty space 23 for receiving the disinfectant fluid. To withdraw the substituate, the housing body 21 has an opening 25 which can be closed by means of a closure element (not shown in FIG. 2). At the outer end of the inner pipe portion 22 there is a connection 26 for the fluid connection 15a leading to the substituate source 16 or to the disinfectant fluid container 18 (FIG. 1).

A suitable connector 27 can be inserted into the opening 25 to withdraw the substituate. The connector 27 has an inner pipe portion 28A which extends into the empty space and which is connected in a fluid-tight manner to the inner pipe portion 22 of the housing body 21 when the connector 27 is connected. The inner pipe portion 28A is surrounded by a touch guard 28B. The opening formed by the inner pipe portion 28A and the opening formed by the touch guard 28B are not in one plane, but are spaced apart from one another such that touching the inner pipe portion 28A of the connector 27 is made difficult or impossible. The connection point 29 between the pipe portion 22 of the housing body and the pipe portion 28A of the connector 27 is located approximately in the centre of the empty space 23.

The disinfectant flows into the empty space 23 via the connection 26, which is connected to the disinfectant container 18. The disinfectant drains out via the channel 38b, which is connected to the drain line or return line 19 (FIG. 1). The disinfectant that has drained or been displaced from the empty space 23 can be collected in a further container (not shown in FIG. 1) and then disposed of, or it can be discarded via a drain.

For better removal of the disinfectant from the empty space 23, sterile air can be directed into the empty space via an opening 38A. The sterile air is compressed by a compressor, for example, and directed into the empty space 23. This compressed air can be used to displace fluid that is present from the empty space, for example to an opening 38B.

The access system P has a measuring electrode 30. In the present embodiment, the measuring electrode 30 is a pin that is electrically insulated from the housing body 21. The pin-shaped measuring electrode 30 is seated in a receiving piece 31 made of an insulating material (for example PEEK), which piece is inserted into the housing body 21. One end of the pin-shaped measuring electrode 30 extends into the empty space 23, while the other end extends out of the housing body 21 to connect an electrical line.

The measuring electrode 30 is arranged such that it interacts with at least one counter electrode 31, 32 via the empty space 23. At least one part of the inner pipe portion 22 acts as the first, inner counter electrode 31, while at least one part of the outer pipe portion 24 acts as the second, outer counter electrode 32. For this purpose, at least one part of the inner pipe portion 22 can consist of a conductive material or at least one part of the outer wall of the inner pipe portion 22 can be provided with a coating 22A made of a conductive material. Correspondingly, at least one part of the outer pipe portion 24 can consist of a conductive material or at least one part of the inner wall of the outer pipe portion 24 can be provided with a coating 24A made of a conductive material. In the present embodiment, the outer wall of the inner pipe portion 22 is provided with a coating 22A, and the inner wall of the outer pipe portion 24 is provided with a coating 24A made of a conductive material.

The monitoring system 2 has a means 33 for generating an electrical signal, and an evaluation and arithmetic means 34, which are shown schematically in FIG. 3 together with the access system P and the patient access 35. The inner pipe portion 22 and the outer pipe portion 24 of the access system P are only indicated in FIG. 3.

The means 33 for generating an electrical signal comprises a controllable frequency generator 33A, which generates an alternating voltage signal V_{ac} at a specified frequency, for example a sinusoidal signal at a frequency of 20 kHz. The frequency generator 33A can be controlled by a control device (CPU1). The alternating voltage can be generated, for example, by means of a VCO (voltage controlled oscillator) or an adjustable signal generator. The CPU1 can be designed, for example, as a programmed microcontroller.

The means 33 for generating an electrical signal and the evaluation and arithmetic means 34 are connected to the measuring electrode 30 via an electrical connecting line 35. To interrupt the electrical connection, a first switch 36 is provided which can be opened or closed by a control signal en_meas from a second control device (CPU2). In addition, a reference resistor R_{Ref} is provided, which establishes a connection between the connecting line 35 and earth when a second switch 37 is closed. The second switch 37 can be opened or closed by a control signal set_ref from the CPU2. The reference resistor R_{Ref} is used to check the operation of the circuit, which will be described below.

For safety reasons, a coupling capacitor C is provided in the connecting line 35, which capacitor can be designed as a Y capacitor. Y capacitors offer high dielectric strength and reliably prevent a breakdown of the capacitor and thus dangerous voltages of the measuring electrode.

According to the invention, an electrical signal is applied to the measuring electrode 30. This can be any voltage having any voltage curve, in particular an alternating voltage. If a conductive path between the measuring electrode 30 and the counter electrode 31, 32 is produced by a fluid residue, a current flow in the current path between the measuring electrode and the counter electrode or a voltage drop across the resulting resistance between the measuring electrode and the counter electrode can be measured.

In order to avoid leakage currents, the pipe portion 22 (or 22A) acting as the at least one counter electrode 31 is earthed, which is shown in FIG. 3. If a plurality of counter electrodes are used, for example as in FIG. 2 (24 or 24A), these are also earthed. In the event of faulty or interrupted earthing, which is indicated in FIG. 3, relatively high leakage currents can occur, which can endanger the patient P. This must be avoided at all costs.

During a dialysis treatment, the substituate flowing in the fluid connection 15a and the substituate line 15b, which substituate contains conductive ions, establishes a conductive fluid connection directly to the vascular system of the patient. In patients who have a central venous catheter as the access to their vascular system, for example in acute dialysis, the catheter is in the immediate vicinity of the heart in order to ensure sufficiently high blood flows in the extracorporeal blood circuit. For these patients in particular, high leakage currents, which could occur via capacitive couplings between the dialysis machine and fluid paths in the patient, must be avoided at all costs.

Increased leakage currents can occur in the event of interruption in the earth connection of the counter electrode 31, as indicated in FIG. 3. A leak at the connection point 29 of the inner housing-side and the inner connector-side pipe portion 22, 24 can lead to a conductive fluid connection between the measuring electrode 30 and the patient's vascular system, which results in a current iₚ. The higher this current, the worse the earth connection of the counter electrode.

FIG. 4 shows an electrical equivalent circuit diagram to illustrate this relationship. The total current i is limited by the internal resistor Rᵢ of the source and the parallel connection of the series-connected individual resistors (impedances) Z_{scl}+Z_{gnd} and Z_{sc2}+Z_{sub}+Zₚ. In the present example of FIG. 4, Rᵢ results from R_{fb} (FIG. 7) and the output resistor (not shown) of an operational amplifier OP1 at the input of the evaluation and arithmetic means 34, which is described in detail below. The coupling capacitor C is disregarded or is dimensioned such that it has no relevant influence. Z_{scl} is the resulting impedance of a conductive bridge between the measuring electrode and the counter electrode. Z_{gnd} is the resulting impedance, which can be assumed here to be a purely ohmic cable connection, of the electrical connection between the counter electrode and the protective conductor PE. Z_{sc2} is the impedance that results from a conductive bridge between the measuring electrode 30 and the possible leakage point in the port, for example at the connection point 29 of the inner housing-side or connector-side pipe portions. Z_{sub} is the impedance of the conductive fluid connection within the substituate line, which connection depends on the length and the diameter of the substituate line 15b (hose line) and on the ion content of the substituate. Zₚ is the resulting impedance between the exit point of the substituate in the patient's vascular system and the patient's earthing, which depends, for example, on the patient's position and build or on the patient's clothing. For example, the patient could touch an earthed metal body, etc. The above-mentioned variables can be complex variables.

The current iₚ, and in particular its magnitude, is a critical risk factor for the patient. If the earth connection Z_{gnd} is faulty, i.e. if the left-hand current path in FIG. 4 is interrupted, the current i no longer branches into two paths, but instead flows exclusively through the right-hand path and thus through the patient. It has to be ensured that the current iₚ does not exceed a magnitude of 50 μA (effective) in order to exclude health risks even in the event of a fault, i.e. an interrupted earth connection of the counter electrode. According to the invention, increased leakage currents can be prevented by the following measures, which can be used individually or in combination.

The means 33 for generating the excitation voltage V_{ac} can be configured such that the magnitude of the excitation voltage V_{ac} is limited such that a leakage current greater than 50 μA does not flow even in the event of a fault.

In addition, the means 33 for generating the excitation voltage V_{ac} can be configured such that pulse-like measurements are carried out. The excitation voltage V_{ac} is only applied for a short period, after which it is switched off in order to be applied again periodically. On average, the result is a current that is smaller than when the excitation voltage is continuously applied.

FIG. 5 shows the time curve of the sinusoidal excitation voltage V_{ac} at a frequency of 20 kHz. The excitation voltage V_{ac} is applied in the time interval Tₒₙ. To apply the alternating voltage, the CPU1 generates a control signal en_meas such that the first switch 36 is closed.

The effective leakage current I_{peff} is calculated using the following equation.

I_{peff}=iₚ\sqrt{Ton/Ttotal}

The time ratio Tₒₙ/Tₜₒₜₐₗ is specified such that the signals can be evaluated, safety is not endangered by excessive “timeouts” and the effective leakage current I_{peff} remains below the limit value.

Furthermore, the means 33 for generating the excitation voltage V_{ac} can be configured such that a minimum frequency for the excitation voltage V_{ac} is specified. FIG. 6 shows that the resulting impedance of the right-hand current path (FIG. 4), which is of crucial importance for the level of the leakage current, rises with increasing frequency.

Consequently, the attenuation D of the signal also increases as the frequency f increases. The excitation frequency is selected depending on the boundary conditions and on the damping behaviour shown in FIG. 6 such that the limit value of the leakage current, for example 20 kHz, cannot be exceeded even in the event of a fault.

The evaluation and arithmetic means 34 has a circuit for measuring and processing the measurement signal. FIG. 7 shows an embodiment of this circuit, which includes three stages A1, A2, A3, each having an operational amplifier OPI, OP2, OP3.

The first stage A1 works as a buffer using the feedback resistor R_{fb}. The electrical signal V_{ac} (alternating voltage) generated by the means 33 is applied to the + input of OP1. The measuring electrode 30 is connected to the − input of OP1 via the coupling capacitor C. The impedance Z_{sc} (short circuit) is a conductive bridge due to fluid or moisture between the measuring electrode 30 and the counter electrode 31, 32, which in this example is at the reference potential PE, i.e. protective earth. This sets the characteristic current i_{sc}. The fluid or moisture that is to be detected generally does not represent a purely ohmic resistance, but rather a mixed ohmic and reactive impedance (capacitive or inductive). The above-mentioned variables can therefore be complex variables. As a result, the current iso is generally out of phase with the alternating voltage V_{ac}. In one embodiment, this can be used not only to detect the presence of fluid or moisture in the port, but also to draw conclusions about the type of fluid. Blood, for example, has a characteristic complex resistance that is different from, for example, water.

If there is no conductive bridge between the measuring electrode 30 and the counter electrode 31, 32, no current iso flows either. In this case, the same voltage is present at the − input of OPI through the feedback resistor R_{fb} as at the + input, i.e. Vac. Since no current then flows through R_{fb} (the input resistance of Al can be considered to be infinitely high to a good approximation) the output voltage of OP1=V_{ac} too.

If, however, a current i_{sc} flows due to fluid or moisture, R_{fb} and Z_{sc} form a voltage divider from the output of the OPI to the PE reference potential (the influence of the coupling capacitor C1 and the measuring electrode can be disregarded in the operating frequency range). As a result, the voltage at the − input of OPI would decrease, but OP1, in its capacity as a differential amplifier fed back via R_{fb}, increases the voltage at the output to such an extent that the + and − input of OPI have the same voltage. The sum of the voltage of V_{ac} and i_{sc}*Z_{sc} is thus set at the output of OPI. This voltage or the transient properties of the voltage are characteristic of moisture occurring in the port, which creates a conductive connection between the measuring electrode and the counter electrode. In step A2 this voltage is rectified and averaged or smoothed, and in step A3 the measurement voltage is amplified. Rectifiers and amplifier circuits can be found in the prior art. The result is a voltage Vade that can be digitised by means of an analogue-digital converter (not shown).

The evaluation and arithmetic means 34, which can include a controller CPU1 (FIG. 3), for example, is configured such that the measurement signal is evaluated using the arithmetic operations described below, in order to identify whether a fluid or moisture is located in the empty space and/or what fluid is located in the empty space. The algorithms known to a person skilled in the art can be used for this purpose. If a fluid or moisture located in the empty space 23 is inferred, the evaluation and arithmetic means generates a control signal or reporting signal.

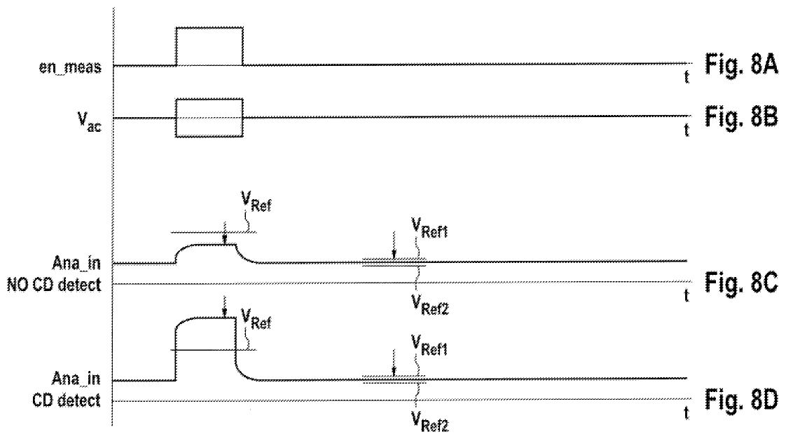

FIG. 8A to 8D show the time curve of the signals. The CPU1 generates the signal en_meas such that the first switch 36 (FIG. 3) is closed (FIG. 8A). At this point in time, the excitation voltage V_{ac}, for example an alternating voltage at a frequency of 20 kHz, is generated (FIG. 8B). The signals in FIGS. 8C and 8D are each characteristic of the resulting voltage Ana_in, which is evaluated by means of the CPU2.

FIG. 8C shows the case where the port is dry (NO CD detect) and FIG. 8D shows the case where a conductive connection has formed between the measuring electrode and the counter electrode (CD detect) due to moisture. In the second case, the resulting voltage Ana_in is higher, which can accordingly be detected by a comparison with a reference value V_{Ref}. In the present example, the measured voltage is A/D converted and the voltage Ana_in is compared with the reference value V_{Ref} in the CPU1 (controller). However, it is also possible to evaluate the resulting voltage Ana_in using a simple (analogue) comparator. If an excitation signal is not present and the measuring electrode 30 is not conductively connected to the circuit, a voltage cannot be measured either, and this can also be checked by the circuit.

The first switch 36, which is controlled by the en_meas signal, is preferably open in the time intervals in which no excitation voltage V_{ac} is intended to be applied to the measuring electrode. As a result, the measuring electrode 30 is isolated from the circuit, and therefore unwanted leakage currents are prevented.

After the interruption of the current path to the coupling capacitor C by opening the first switch 36, and after connecting the reference resistor R_{ref} by closing the second switch 37 (en_meas=off, set_ref=on), an expected value for the voltage Ana_in can be checked. If the measured value deviates from the expected value, there is an error. In FIGS. 8C and 8D, an upper and a lower reference value V_{Ref1}, V_{Ref2} are shown. For example, it can be checked whether the voltage is between the upper and the lower reference value V_{Ref1}, V_{Ref2}.

On the basis of the level of the voltage Ana_in or an electrical variable that correlates with the voltage, it can also be determined whether and to what extent the empty space is filled with fluid. This is particularly advantageous for checking the disinfection process.

The inner pipe portion 22 or the outer pipe portion 24 can act as the counter electrode 31, 32. For checking the fill level, at least one part of the outer pipe portion 24 can alternatively or additionally be designed as the counter electrode 32, for example particular regions of the inner wall of the outer pipe portion 24 can be provided with a conductive coating 24A, it being possible for a plurality of current paths to form from the measuring electrode to the individual regions. Then, depending on the fill level of disinfectant fluid in the empty space, a different resistance is set, further current paths being formed as the fill level of the empty space increases, so that the resistance decreases, and this can be identified using the evaluation and arithmetic means 34. If a plurality of counter electrodes is provided, the evaluation and arithmetic means 34 can also be configured such that a plurality of measurement signals can be evaluated. Depending on the fill level, voltage values or current values are obtained for the individual counter electrodes, which values can be compared with reference values that are characteristic of the particular fill level.

In the embodiments described above, a substantially rectified signal Ana_in is evaluated, as a result of which the information on the phase shift between the measurement signal and the excitation signal is lost. However, it is also possible to evaluate a non-rectified alternating voltage signal/alternating current signal. If the measurement is not only carried out at an excitation frequency, but also said frequency is varied (frequency sweep), characteristic curves result, which can be converted into impedance curves (magnitude of impedance as a function of frequency), for example. For blood, for example, the structure (cells in plasma) results in specific current paths and corresponding impedances depending on the measurement frequency. In this regard, express reference is made to DE 10 2010 028 902 A1 and in particular to FIGS. 1 to 4 thereof and the associated description of the figures. By the method known from DE 10 2010 028 902 A1 and using the monitoring system 2 according to the invention, it is therefore possible to determine what the fluid is. Due to their composition (without cells), dialysate or substituate have different characteristic impedance curves and can also differ from one another, for example, in terms of ion density, i.e. the density of the free charge carriers.

1. Access system for a medical device, which system has a housing body in which an inner pipe portion for transporting a medical fluid is formed, which portion is enclosed by an outer pipe portion so as to form an empty space for receiving a disinfectant fluid, the housing body having an opening that can be closed by a closure element, wherein
a measuring electrode and at least one counter electrode are arranged in the housing body such that the measuring electrode interacts with the at least one counter electrode via the empty space.

2. The access system according to claim 1, wherein the measuring electrode is a pin which is electrically insulated from the housing body and extends into the empty space.

3. The access system according to claim 1, wherein the at least one counter electrode is formed by at least one part of the inner pipe portion.

4. The access system according to claim 3, wherein at least one part of the inner pipe portion consists of a conductive material or at least one part of the outer wall of the inner pipe portion (22) is provided with a coating made of a conductive material.

5. The access system according to claim 1, wherein the at least one counter electrode is formed by at least one part of the outer pipe portion.

6. The access system according to claim 5, wherein at least one part of the outer pipe portion consists of a conductive material or at least one part of the inner wall of the outer pipe portion is provided with a coating made of a conductive material.

7. The access system according to claim 1, wherein the access system comprises a connector which can be inserted into the opening and which has a pipe portion that extends into the empty space and can be connected to the inner pipe portion of the housing body in a fluid-tight manner, the connection point between the inner pipe portion of the housing body and the pipe portion of the connector being located in the empty space.

8. A monitoring system comprising the access system according to claim 1, wherein the monitoring system has a means for generating an electrical signal, which means is electrically connected to the measuring electrode and to the at least one counter electrode, and in that the monitoring system has an evaluation and arithmetic means which is configured such that a current flowing between the measuring electrode and the at least one counter electrode or a voltage applied between the measuring electrode and the at least one counter electrode is evaluated.

9. The monitoring system according to claim 8, wherein the evaluation and arithmetic means is configured such that a current flowing between the measuring electrode and the at least one counter electrode or a voltage applied between the measuring electrode and the at least one counter electrode is evaluated such that the presence or absence of a fluid or moisture in the empty space is inferred.

10. The monitoring system according to claim 9, wherein the evaluation and arithmetic means is configured such that a control signal or reporting signal is generated if a fluid or moisture located in the empty space is inferred and/or a control signal or reporting signal is generated if a fluid or moisture located in the empty space is not inferred.

11. The monitoring system according to claim 8, wherein the evaluation and arithmetic means is configured such that a current flowing between the measuring electrode and the at least one counter electrode or a voltage applied between the measuring electrode and the at least one counter electrode is evaluated such that a conclusion is made as to whether a particular fluid is present in the empty space.

12. The monitoring system according to claim 8, wherein the means for generating an electrical signal is configured such that an electrical signal is generated at successive time intervals.

13. The monitoring system according to claim 8, wherein the means for generating an electrical signal has a frequency generator for generating an alternating voltage signal or alternating current signal.

14. The monitoring system according to claim 13, wherein the evaluation and arithmetic means has a means for rectifying an alternating voltage signal, the evaluation and arithmetic means being configured such that the rectified alternating voltage signal is compared with a reference value, the presence of a fluid or moisture in the empty space being inferred if the rectified alternating voltage signal deviates from the reference value.

15. A medical treatment device having the monitoring system according to claim 8.

16. The medical treatment device according to claim 15, wherein the medical treatment device is a blood treatment apparatus having an extracorporeal blood circuit, which apparatus has a means for providing substituate, the inner pipe portion being in fluid communication with the means for providing substituate.

17. A method for monitoring an access system for a medical device, which system has a housing body in which an inner pipe portion for transporting a medical fluid is formed, which portion is enclosed by an outer pipe portion so as to form an empty space for receiving a disinfectant fluid, the housing body having an opening that can be closed by a closure element, wherein by means of a measuring electrode which interacts with the at least one counter electrode via the empty space, an electrical signal is coupled in, a current flowing between the measuring electrode and the at least one counter electrode or a voltage applied between the measuring electrode and the at least one counter electrode being evaluated such that the presence or absence of a fluid or moisture in the empty space is inferred and/or a conclusion is made as to whether a particular fluid is present in the empty space.

18. The method according to claim 17, wherein the measuring electrode is a pin which is electrically insulated from the housing body and extends into the empty space.

19. The method according to claim 17, wherein the at least one counter electrode is formed by at least one part of the inner pipe portion and/or is formed by at least one part of the outer pipe portion.

20. The method according to claim 17, wherein the electric signal is coupled in at successive time intervals.

21. The method according to claim 17, wherein the electrical signal is coupled in via a coupling capacitor.

22. The method according to claim 17, wherein the electrical signal is an alternating voltage having a specified frequency, or the electrical signal is an alternating voltage having a frequency that changes over time (frequency sweep).
